(19) United States
(12) Patent Application Publication
(10) Pub. No.: US 2019/0159740 A1
(43) Pub. Date: May 30, 2019

(54) METHOD FOR GENERATING A RADIATION IMAGE OF A REGION OF INTEREST OF AN OBJECT

(71) Applicant: Agfa HealthCare NV, Mortsel (BE)

(72) Inventor: Cis VAN AMMEL, Mortsel (BE)

(21) Appl. No.: 16/092,773

(22) PCT Filed: Apr. 7, 2017
(86) PCT No.: PCT/EP2017/058382

§ 371 Date: Oct. 11, 2018

(30) Foreign Application Priority Data
Apr. 15, 2016 (EP) 16165462.9

(51) Int. Cl. A61B 6/03; A61B 6/00; G06T 11/00; G06T 1/00; G06T 7/00

(52) U.S. Cl. CPC A61B 6/032; A61B 6/467; G06T 11/008; G06T 2211/40; G06T 7/0012; G06T 2207/10081; G06T 2207/30004; G06T 1/0007

(57) ABSTRACT

A region of interest first displayed on a model image of an object is mapped onto a corresponding location on the object defined relative to determined locations of predefined features. A source of radiation is set so that the region of interest on the object is irradiated.

### CROSS REFERENCE TO RELATED APPLICATIONS

This application is a 371 National Stage Application of PCT/EP2017/058382, filed Apr. 7, 2017. This application claims the benefit of European Application No. 16165462.9, filed Apr. 15, 2016, which is incorporated by reference herein in its entirety.

### BACKGROUND OF THE INVENTION

### 1. Field of the Invention

The present invention is in the field of computed and digital radiography and more specifically relates to a method of recording of x-ray image of a region of interest (ROI) of a patient, an animal or an object.

A particular application of this invention relates to the recording of a radiation image of a long length object such as a full leg or a full spine by means of partial radiation images which together form the radiation image of the long length object. The partial images can be defined as regions of interest to be irradiated.

### 2. Description of the Related Art

In radiography, a radiation image of long length object may have to be taken, such as an image of an entire spine or of a leg or of a large part of these objects.

In Computed Radiography (CR), x-ray images of long length objects are recorded on a set of Imaging Plates (IP) which are placed in a fixed positional relationship in a holder and which partially overlap each other. A long length image is created by exposing the assembly to a radiation image of the long length object and by reading out the partial mages from the set of imaging plate. Next, these partial images are combined into a full leg-full spine image.

The source of radiation (x-ray source) is adjusted so as to irradiate all imaging plates that are present in the holder simultaneously.

Accurate alignment and measurement can be obtained by superimposing an object of known geometry such as a grid of x-ray attenuating material covering the region to be imaged. The image of the grid which is superimposed on the x-ray image of the long length body can be used for correcting and aligning the partial images to reconstruct the object of known geometry (see EP0919858, EP0866342). This technique does not suffer from patient movement since all images are acquired in a single X-ray exposure.

In recent years, Digital Radiography (DR) has become a valuable alternative for CR. In DR, flat panel detectors (FPD) are used which are more costly than the IP's for CR, so an alternative to the one-shot long length imaging technique of CR using a multiplicity of CR detectors is needed. This is achieved by taking plural partial images by moving the position of the FPD while turning the X-ray tube or moving the X-ray tube parallel to the FPD thereby and pasting the partial images to obtain a composite long length image.

In both above-described techniques at least one region of interest (and in most cases more than one region of interest) to be exposed is to be defined.

Conventionally a patient is positioned on a so-called wall stand. The operator selects the appropriate examination type on a workstation. Top and bottom values of the region to be exposed are entered manually into the software controlling the x-ray image recording application running on a workstation. The software then determines the top and bottom values of individual regions of interest pertaining to partial images to be generated. The source and detector are then adjusted so as to sequentially record the partial images which together form the entire long length image.

For a Full leg/full spine application, the above described top and bottom values of the region to be exposed are read on a stitching grid used for stitching partial images that together form the complete full leg/full spine image and input into the software.

The above described techniques may suffer from inaccuracy and may be inconvenient to implement.

The problem to be solved has been explained with respect to the situation in which at least one region of interest is to be determined in the context of generating a radiation image of a long length body.

However, the need for an accurate method to expose a region of interest exists also in other types of applications.

### SUMMARY OF THE INVENTION

It is thus an aspect of the present invention to further improve the technique for accurately determining and irradiating a region of interest in a radiographic recording process.

The above-mentioned aspects are obtained by a method as set out below. Specific features for preferred embodiments of the invention are also set out below.

In general a method for irradiating a region of interest according to the present invention comprises the steps of
defining said region of interest and displaying it on a model image of the object,
determining the location of predefined features on said object,
locating on the object the region of interest corresponding with the region of interest displayed on said model image, on the basis of the determined locations,
adjusting a source of radiation so that it emits radiation within said region of interest and generates a radiation image of said region of interest,
detecting said radiation image by means of a radiation detector,
reading out the radiation image stored in said radiation detector.

The present invention is applicable to irradiation of a region of interest on an object. This object may be a human body, e.g. of a patient. Likewise the method is applicable to the irradiation of a region of interest on an animal or an object. Whenever in the context of this application reference is made to an object this is to be understood as not being limited to irradiation of an object but also being applicable to the irradiation of a region of interest on a human patient, an animal or object.

The invention is much more convenient for the user that the prior art method in which top and bottom values of a region of interest were to be entered manually into a software running on a workstation. In a specific embodiment the operator does not have to perform measurements (e.g. on the stitching grid as has been described higher). Furthermore in a specific embodiment the user does not have to leave the workstation to perform measurements on the actual location of the region of interest.

A defined region of interest is first displayed on a model image of an object to be irradiated.

The model image can be either a 3D or a 2D image. The model image suits to visualize the region of interest which is envisaged to be irradiated relative to a model of an object (e.g. a patient). This model image is preferably stored in advance in a memory of a workstation or in a radiology information system and can be retrieved whenever a x-ray exposure is to be performed.

In one embodiment the region of interest displayed on the model image can be defined by means of its borders, e.g. horizontal top and bottom lines. It is furthermore possible to define the region additionally by vertical left and right border lines.

In a specific embodiment a model image of a patient (or animal or object) is displayed and on top of this model image a number of bars defining the borders of the region of interest are displayed.

In one embodiment the operator can implement minor adaptations to the borders of the region of interest by moving these bars on the screen in horizontal or vertical direction.

The provision of these bars is optional. In a more general embodiment these bars are not provided.

Once the region of interest is defined relative to the model image, it is then brought in relation with the actual position of the object.

The actual position of the object is defined by means of actual geometric information regarding to the object.

In the context of the present invention actual geometric information is obtained by determining the location of predefined features on the object.

The type of predefined features that is used depends on the type of object and the specific radiographic examination.

Suitable features are features the position of which can be easily measured or determined.

An example of such features are joints in the human body.

Preferably the location of predefined features is measured or derived from an actual image of the object or at least of a part of the object comprising the region of interest and being located in the actual position for x-ray image recording.

The actual image can be a 3D image generated by a 3D camera.

Alternatives are possible.

For example a 2D image together with data on the distance between the object (patient)'s position and the detector or the source of radiation can be used as information on the actual position of the object (patient).

In one embodiment an image is used that represents the actual contour of the object (patient) as well as the distance between object (patient) and detector or source of radiation.

In a next step the region of interest corresponding with the region of interest displayed on the model image, is located on the object on the basis of the determined locations of the predefined features by mapping corresponding locations onto each other.

Mapping the information determined on the model image to the actual position can be done in different ways. In one embodiment the position of the predefined features (also called landmarks) on the image are determined and mapped onto corresponding landmarks on the actual image, e.g. the joints in the human body on a model image of the human body are mapped onto corresponding positions of the joints on the actual image.

Alternatively sensors can be used to assist the mapping. These sensors may be coupled to the patient or alternatively to e.g. the exposure table or exposure stand on which the patient is positioned.

The sensors provide reference information on the actual object position and can be used to guide the mapping.

Still other alternatives are possible, it is important that the actual position of the patient can be identified and that reference points can be found which may assist in mapping the ROI borders on the model image to the actual image of the object/patient in his actual position.

Finally the source of radiation is adjusted so as to irradiate the region of interest on the object, whereby this region of interest is defined by the mapped locations on the actual image of the object.

The radiation image of the irradiated region of interest is detected by a digital radiation detector and is finally read out.

In a specific application the above method steps are applied in the context of recording a radiation image of a long length object. The method then comprises the steps of defining a region of interest, generating partial radiation images of said long length object within said region of interest, reading out said partial images so as to obtain digital signal representations of said partial images and combining the digital signal representations of said partial images to form an image of said long length body.

In this context a long length object refers to an object the radiation image of which or of part of which cannot be recorded on a single CR radiography detector or a DR radiography detector in a single exposure. Either partial images are generated during one exposure on a set of radiography detectors or more than one exposure is made of different parts of the object so that the corresponding radiation images together form the radiation image of the long length object.

Already at the time when examination types are configured, the corresponding points defining the region of interest can be indicated on the model image and can be associated with the examination type.

Simple selection of an examination type by means of a name associated with the examination type allows the operator to retrieve the pre-defined coordinates of points defining the region of interest for this type of examination.

These theoretically defined points can then be adapted to correspond to the real time situation.

The method of the present invention is advantageous in that it is a fast method which can be performed with minimal user interaction and which avoids errors when positioning the x-ray source to generate one or more x-ray images of the long object or of parts of the long object.

Further advantages and embodiments of the present invention will become apparent from the following description.

### BRIEF DESCRIPTION OF THE DRAWINGS

FIG. 1 is an example of a set up in a radiology room which is suitable for performing the method of the present invention.

FIG. 2 illustrates the display of borders defining the region of interest on the model image.

### DETAILED DESCRIPTION OF THE PREFERRED EMBODIMENTS

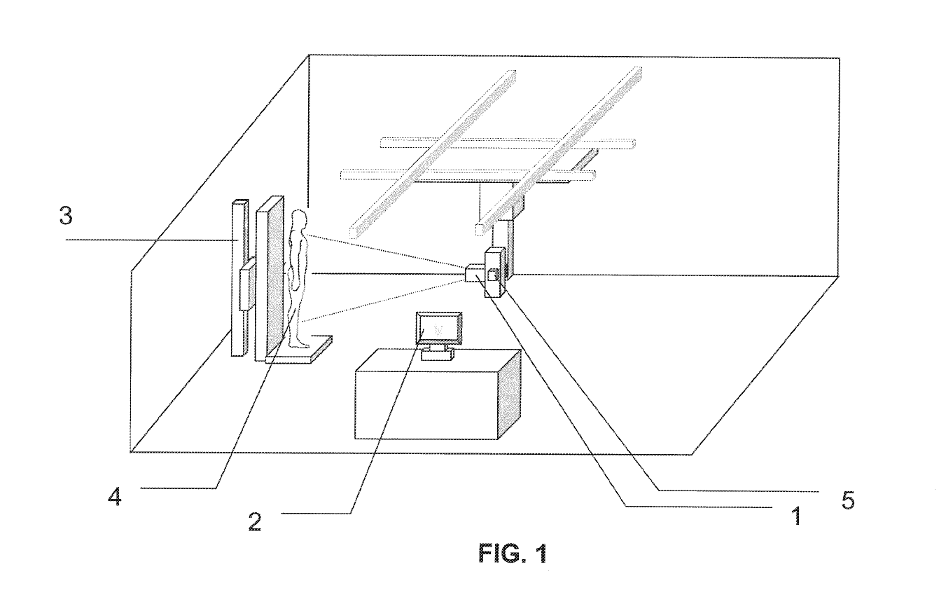

### System (FIG. 1):

The present invention can be applied in the context of a radiology room in which a radiographic image of an object, a human patient or an animal is acquired.

The invention will be explained with reference to a human patient but is also applicable to an animal or a material object.

A radiology room generally is equipped with a source of radiation (1) coupled to a workstation (2) running software for patient identification as well as examination type identification.

The workstation can be coupled to a radiology information system (RIS) so as to be able to retrieve i.a. stored patient demographic data or examination type data from the RIS and display and use these data.

The operational parameters for the source of radiation can be set in correspondence with the examination type that is selected on the workstation.

These operational parameters comprise kV, MAS, source-object distance etc.

The setting of the source of radiation in correspondence with these operational parameters can be controlled from the workstation. Alternatively an additional console controlling the settings of the source of radiation can be provided.

In the radiology room a patient support (3) is provided onto which the patient (4) is positioned when the radiographic image is taken.

This patient support may be a supporting table onto which the patient is lying during image recording. Alternatively the patient support may be part of a wall stand, in this case the patient stands in upright condition on the patient support during image recording.

In accordance with the present invention the radiology room further is equipped with means (5) for generating a 3D image of the patient to be examined. These means can be a 3D camera directed to the location where the object is positioned. For example the 3D camera can be adhered to the source of radiation so that when the source of radiation is directed towards the patient the camera is in optimal position to make a so-called actual image of the patient, i.e. an image of the patient in his actual position in the radiology room.

The output of the camera for generating an actual image is fed into the workstation so that this image can be visualised on the display screen of the workstation.

In some applications such recording of a radiation image of a long length object such as a full spine or a full leg, the patient support is not only used for supporting a patient but likewise serves to identify (a) border(s) of (a) region(s) of interest. This may be performed by identifying the patient's position relative to a ruler provided on the patient support next to the patient. This ruler can be omitted since the indications on the patient position can be derived from the actual image of the patient that is generated. (Not shown)

Method

In accordance with the present invention, prior to radiographic examination a service technician has defined a number of examination types. For each of these examination types borders of one or more regions of interest are specified.

These examination types and the corresponding examination type definitions comprising the location information of at least one region of interest were stored in advance in the workstation or stored in the RIS.

In the case of imaging of a long length object such as a complete spine or a leg, the examination type definition may comprise the locations of the borders of the entire region of interest the radiologist is interested in.

Alternatively or additionally the examination type definition may comprise the location of the partial regions of interest that together form the complete region of interest in which the radiologist is interested.

Once the user selects an examination type, the information of the border of the area(s) of interest is retrieved and is displayed on the display screen of the workstation on top of a model image of a fictitious patient. This model image can be a 3D or a 2D image that is stored in advance in the workstation's memory.

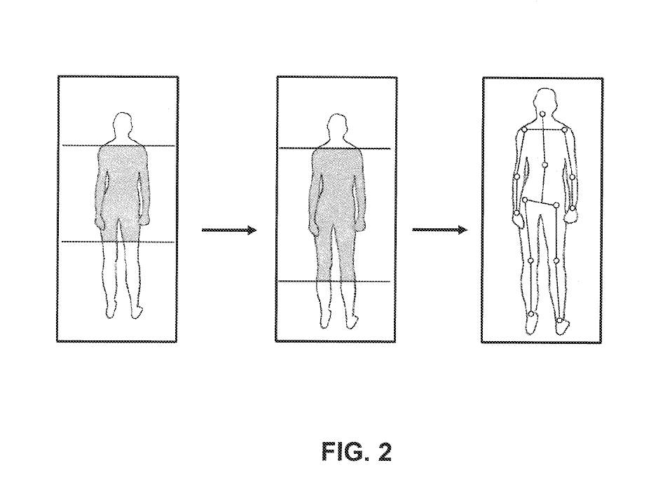

FIG. 2 shows a model image which is displayed on the workstation and shows for a full spine examination the borders of a region of interest on that model image.

The region of interest is delineated by two horizontal lines. This embodiment is only exemplary. It is also possible to delineate the region in another way. Furthermore it is possible to delineate this region of interest not only by means of horizontal borders but also by means of vertical borders so that a rectangular field of interest is delineated.

In one embodiment the user can adapt the region of interest by shifting the border lines in vertical and/or horizontal direction. In the example shown, the user adds the knees to the delineated region of interest.

Once the user is satisfied with the borders, he will confirm these borders on the workstation.

In a next step of the process the ROI (or ROI borders) confirmed on the workstation will be mapped onto an actual image of the patient positioned in front of the x-ray tube (either on a wall stand or on a bucky device).

This actual image can be generated in different ways.

In one embodiment the actual image is recorded by means of a 3D camera provided in the radiology room (for example on an assembly supporting the source of radiation as well as the camera) and directed so as to be able to record an image of the patient.

This position is preferable because in an operational setting the source of radiation as well as the camera are then directed towards the patient.

In another embodiment the actual image is a 2D image and additional information is acquired on the object-source distance.

The actual image (or actual image and object-source distance) is fed into the workstation and displayed on the workstation's display screen.

The above-mentioned mapping of border data from the model image to the actual image can be performed in a number of different ways.

In one embodiment a number of pre-defined features or landmark point defined on the model image are mapped onto corresponding locations on the actual image. For example the position of joints is defined on the model image and corresponding positions on the actual image are determined.

With these data on the borders of the region of interest resulting from this mapping, the system can calculate the settings of the source of radiation that are required to take a radiography of the envisaged region of interest on the long length image.

In the case of a long length image the system may calculate by means of the data on the total region of interest the required settings for the x-ray source in order to be able to generate a number of partial images which together form the entire image of the long length object.

For example when a full leg-full spine image is composed of three partial images acquired by positioning the x-ray source in different positions so as to irradiate different parts of the full leg-full spine body part, this definition may comprise the coordinates of the borders delineating these partial images.

Finally the x-ray source settings are adjusted and the x-ray exposures are made.

In case a full leg or full spine image is recorded on a DR detector partial images are sequentially recorded by each time repositioning the DR detector and or the source of radiation and the DR detector is read out before a next partial image is recorded.

The read out partial images are combined so as to form the entire image of the long length object.

This invention has been explained with regard to a long length image but can be applied to any type of examination in which a region of interest is to be irradiated.

1-6. (canceled)

7. A method of recording a 3D radiation image generated by a 3D camera of a region of interest of an object, the method comprising:
defining the region of interest relative to a model image of the object and displaying the region of interest on the model image;
determining locations of predefined features on the object on an actual image of at least a portion of the object in an image recording position;
mapping onto the actual image of a patient positioned in the image recording position the region of interest corresponding to the region of interest displayed on the model image based on the locations of the predefined features;
adjusting a source of radiation to emit radiation within the region of interest and generate a radiation image of the region of interest;
detecting and storing the radiation image with a radiation detector; and
reading out the radiation image stored in the radiation detector.

8. The method according to claim 7, wherein the model image is a 3D model image.

9. The method according to claim 7, further comprising the step of:
displaying borders of the region of interest on the model image.

10. The method according to claim 9, further comprising the step of:
adjusting the borders of the region of interest displayed on the model image.

11. The method according to claim 7, wherein the region of interest represents a portion of an elongated image, and a partial radiation image is generated by irradiating the region of interest.

12. The method according to claim 7, wherein the object is a patient.
